(19) United States
(12) Patent Application Publication
(10) Pub. No.: US 2024/0207431 A1
(43) Pub. Date: Jun. 27, 2024

(54) USE OF ANTIBODY-DRUG CONJUGATE IN COMBINATION WITH IMMUNE CHECKPOINT INHIBITOR IN TREATMENT OF UROTHELIAL CANCER

(71) Applicant: REMEGEN CO., LTD., Yantai (CN)

(72) Inventors: Jianmin FANG, Yantai (CN); Jing JIANG, Yantai (CN); Shenjun LI, Yantai (CN); Xiaohong SU, Yantai (CN)

(21) Appl. No.: 18/511,857

(22) Filed: Nov. 16, 2023

Related U.S. Application Data
(63) Continuation of application No. PCT/CN2022/093631, filed on May 18, 2022.

(30) Foreign Application Priority Data
May 21, 2021 (CN) 202110559728.8

(51) Int. Cl. A61K 47/68; A61K 39/395; A61P 35/00

(52) U.S. Cl. CPC A61K 47/6851; A61K 39/3955; A61K 47/68031; A61K 47/6889; A61P 35/00

(57) ABSTRACT

Provided is use of an antibody-drug conjugate targeting Her2 in combination with an immune checkpoint inhibitor such as PD-1 antibody or PD-L1 antibody, in the preparation of a medicine for treating patients with urothelial cancer, especially locally advanced or metastatic urothelial cancer. Compared to treatment with either of the two drugs alone, treatment with both in combination has a marked synergistic effect and significant therapeutic effect. In addition, the combination treatment had good efficacy for patients with low HER2 IHC expression (1+).

### CROSS REFERENCE TO RELATED APPLICATIONS

This application claims the priority benefit of Chinese Application Nos. 202110559728.8, filed May 21, 2021, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761682008641SEQLIST.txt, date recorded: May 16, 2022, size: 27,948 bytes).

### FIELD

The present disclosure relates to the field of precise treatment of cancers, to use of an Antibody Drug Conjugate (ADC) targeting HER2 (Human Epidermal Growth Factor Receptor 2) in combination with an immune checkpoint inhibitor in the treatment of urothelial cancer.

### BACKGROUND

Urothelial carcinoma (UC; or Transitional cell carcinoma, TCC) is a type of cancer that commonly occurs in the urinary system: kidneys, bladder, and accessory organs. It is the most common type of bladder cancer, as well as ureteral, urethral and urachal cancers. It is the second most common type of renal cancer, accounting for 5-10% of all primary renal malignancies. (en.wikipedia.org:https://en.wikipedia.org/wiki/Transitional, cell carcinoma).

The urothelium (also known as transitional epithelium) is the inner side of the bladder, ureters and urethra, and the lining of the renal pelvis (the part of the kidney where urine is collected). It consists of urothelial cells and transitional cells. These cells can turn into cancer cells, i.e., known as urothelial cancer (or transitional cell carcinoma).

Depending on the invasiveness of the cancerous cells, urothelial cancer can be non-invasive (in the lining of the bladder only) or invasive (growing into other layers of the bladder wall). Of these, non-invasive urothelial cancer grows only in the inner membrane of the bladder and does not grow deeper in the bladder wall. At diagnosis, the tumors in 50-60% of patients with urothelial cancer are non-invasive. Types of non-invasive urothelial cancer include: non-invasive squamous urothelial cancer (also known as carcinoma in situ): non-invasive papillary urothelial cancer, high grade; and non-invasive papillary urothelial cancer with low-grade malignancy, where non-invasive papillary urothelial tumors with low malignant potential are less likely to develop into invasive cancers.

In contrast, invasive urothelial cancer grows from the inner membrane of the bladder into a deeper layer of the bladder wall, such as the connective tissue (known as lamina propria) and the muscle layer (known as muscularis). At diagnosis, the tumors in 40-50% of patients with urothelial cancer are invasive.

In theory, urothelial cancer can start anywhere in the urinary tract, including but not limited to the renal pelvis, ureters, the bladder, or the urethra.

When the relevant tumor cells have not metastasized, surgical resection is the preferred treatment option. For patients with metastatic tumors, anticancer drug treatment is generally required. The current first-line therapy is a combination therapy ofgemcitabine and cisplatin. However, radiation therapy does not work well in urothelial cancer and is generally used as an adjuvant therapy. When treating carcinomas in the renal pelvis/ureter epithelium, BCG injection therapy (catheter injection of Mycobacterium bovis) can be used.

Urothelial cancer metastasizes and frequently recurs. Radical cystectomy is the first choice for patients with tumor involving the muscularis, and strict and regular review is required after the surgery. Therefore, the treatment therefor is difficult and the recurrence rate is high. (Li Xuesong, Wang Gang, and Zhang Qian, eds., Essence of Urology Cases, Peking University Medical Press, 2017). Mitomycin (a chemotherapy drug) administered to the bladder early after the surgery (within 24 hours) as a single dose or several weeks after the surgery as a six-dose regimen is also a treatment option for some patients.

Vinflunine has been approved in Europe for the treatment of urothelial advanced or metastatic TCC (Bellmunt, J. et al., J Clin Oncol. 27(27): 4454-4461 (2009)). Several agents have shown moderate activity with a median survival of 5 to 10 months when tested as single agent therapy (Yafi, F. A. et al., Current Oncol 18(1): e25-e34 (2011)). In the metastatic setting, docetaxel was administered to patients with transitional cell carcinoma as a palliative option (NCCN 2014) Moreover, the US and Canada medical community endorses docetaxel as a treatment regimen for advanced diseases based on an evidence from a Phase 2 study (WO2016/064649A1).

In recent years, new drugs for the treatment of urothelial cancer mainly include:

1. Roche's Atezolizumab (2016), the first anti-PD-L1 cancer immunotherapy drug approved in the European Union, useful for the treatment of metastatic urothelial cancer. The objective remission rate (ORR) of patients assigned to the experimental group was 63%, while the ORR of patients in the chemotherapy group was 21%. Results from a cohort in the IMvigor210 study showed a median overall survival (OS) of 15.9 months in the atezolizumab arm. Common adverse reactions of atezolizumab included fatigue, decreased appetite, nausea, dyspnea, diarrhea (18.6%), fever, rash, vomiting, arthralgia, weakness, and itching.

2. Bristol-Myers Squibb's Nivolumab (2017), approved by the US FDA for patients with locally advanced or metastatic urothelial cancer. Nivolumab is an anti-PD-1 monoclonal antibody. The clinical data showed that the objective remission rate (ORR) was 19.6%, the median duration of treatment was 3.3 months (time range: 0-13.4 months), and 54% of patients experienced serious adverse events. The most common serious adverse events with an incidence of at least 2% included urinary tract infection, sepsis, diarrhea, small bowel obstruction, and deterioration of general health. The most common adverse reactions included fatigue, muscle and bone pain, nausea, and decreased appetite. Nivolumab treatment was discontinued due to adverse reactions in 17% of patients, and dosing was delayed in 46% of patients due to adverse reactions. Treatment-related death occurred in 4 patients due to pneumonia or cardiovascular failure.

3 Johnson & Johnson's Janssen erdafitinib, a fibroblast growth factor receptor (FGFR) tyrosine kinase inhibitor, approved by the US FDA (2018) for the treatment of urothelial cancer. Study results showed that erdafitinib had an objective remission rate (ORR) of 42% in 59 patients with relapsed/refractory metastatic urothelial cancer whose tumors harbored FGFR mutations (Janssen Announces U.S. FDA Breakthrough Therapy Designation for Erdafitinib in the Treatment of Metastatic Urothelial Cancer).

4. Padcev (enfortumab vedotin), approved by the US FDA in December 2019 for patients with locally advanced or metastatic urothelial cancer who had previously received the treatment of a PD-1/L1 inhibitor and had received a platinum-based chemotherapy regimen in neoadjuvant/adjuvant therapy or in the treatment of the locally advanced or metastatic disease. The data showed that Padcev treatment rapidly shrank tumors in most patients, with an objective remission rate of 44% (55/125, 95% CI: 35.1-53.2), a complete remission rate of 12% (15/125), and a median duration of remission of 7.6 months (range: 0.95-11.3+). Padcev is a first-in-class ADC targeting a cell surface protein that is highly expressed in bladder cancer. The drug is prepared by conjugating a human IgG1 monoclonal antibody targeting Nectin-4, enfortumab, with a cytotoxic agent, MMAE (monomethyl auristatin E, a microtubule disrupting agent).

5. An antibody-drug conjugate (i.e., Disitamab vedotin) that can specifically bind to a HER2 target and has a drug moiety being MMAE, disclosed by Chinese patent of publication no. CN105008398A. Currently, the drug is being explored as a treatment for various HER2-expressing (IHC 1+ or above) cancer indications including breast cancer, such as gastric and urothelial cancers, and HER2-low expressing (IHC 2+/FISH− or IHC 1+) cancer indications, such as HER2-low expressing breast cancer. In September 2020, the U.S. FDA also granted a breakthrough therapy designation to Disitamab vedotin for the second-line treatment of HER2-expressing (IHC 2+ or IHC 3+) locally advanced or metastatic urothelial carcinoma indication.

At present, the ORR of the first-line chemotherapy for metastatic urothelial cancer (mUC) is approximately 50%, and the ORR of Enfortumab vedotin combined immunization for the first-line treatment of platinum-intolerant patients (EV-103 study) was reported to be 73.3% in overseas countries.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

The present disclosure provides methods and uses for treating urothelial cancer patients with an anti-HER2 antibody-drug conjugate (ADC) and an immune checkpoint inhibitor. These methods and uses are based at least in part on the in-depth analysis of animal models and clinical data presented herein, which demonstrate Applicant's surprising discovery that Her2 antibody-drug conjugates (ADCs) and immune checkpoint inhibitors in combination had a synergistic effect in the treatment of urothelial cancer, especially in patients with locally advanced or metastatic urothelial cancer, and clinical benefit compared with the existing standard therapies. The ORR increased with higher expression of HER2 or PD-L1. In addition, for patients with low HER2 IHC expression (1+), the combination treatment still had good efficacy.

For example, the combination of Disitamab vedotin and PD-L1 antibody had a synergistic effect on the proliferation inhibition of HT-29 subcutaneously transplanted tumors. Furthermore, in the clinical trial of Disitamab vedotin in combination with PD-1 antibody (Toripalimab), the results indicated that the combination therapy yielded an improved patient outcome compared to single treatment alone, in particular with respect to PFS.

Provided herein are uses of an antibody-drug conjugate (ADC) in combination with an immune checkpoint inhibitor in the preparation of a medicament for treating a urothelial cancer patient, wherein the antibody-drug conjugate has the structure of the general formula Ab-(L-U)ₙ, wherein Ab represents anti-Her2 (Human epidermal growth factor receptor 2) antibody; L represents a linker; U represents a conjugated cytotoxic molecule; and n is an integer from 1 to 8, and represents the number of cytotoxic molecules bound to each antibody, and wherein: the antibody comprises a heavy chain variable region and a light chain variable region, wherein the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-ve-pAB), wherein the linker is covalently linked to the antibody by means of sulfhydryl conjugation, and the linking site is the interchain disulfide bond site of the antibody: the cytotoxic molecule U comprises MMAE (monomethyl auristatin E); and the immune checkpoint inhibitor is a PD-1 antibody or a PD-L1 antibody.

Also provided herein are methods for treating a urothelial cancer patient, comprising administering to the patient an effective amount of an antibody-drug conjugate (ADC) and an immune checkpoint inhibitor, wherein the antibody-drug conjugate has the structure of the general formula Ab-(L-U)ₙ, wherein Ab represents anti-Her2 (Human epidermal growth factor receptor 2) antibody: L represents a linker; U represents a conjugated cytotoxic molecule; and n is an integer from 1 to 8, and represents the number of cytotoxic molecules bound to each antibody, and wherein: the antibody comprises a heavy chain variable region and a light chain variable region, wherein the CDR of the heavy chain variable region and/or the CDR of the light chain variable region have the same CDR sequences as Disitamab vedotin; the linker L comprises Maleimido-Caproyl-Valine-Citrulline-p-Aminobenzyloxy (mc-ve-pAB), wherein the linker is covalently linked to the antibody by means of sulfhydryl conjugation, and the linking site is the interchain disulfide bond site of the antibody: the cytotoxic molecule U comprises MMAE (monomethyl auristatin E); and the immune checkpoint inhibitor is a PD-1 antibody or a PD-L1 antibody.

In some embodiments of all of the above, the patient is positive for HER2 expression. In some embodiments of all of the above, a sample obtained from urothelial cancer of the patient is HER2 positive. In some embodiments of all of the above, the sample obtained from urothelial cancer of the patient is HER2 positive based on a immunohistochemistry (IHC) assay. In some embodiments of all of the above, HER2 expression in the sample obtained from urothelial cancer of the patient is IHC 3+ or IHC 2+. In some embodiments of all of the above, the patient is positive for PD-L1 or PD-1 expression.

In some embodiments of all of the above, the antibody comprises a heavy chain variable (VH) region and a light chain variable (VL) region, wherein the VII region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8). In some embodiments of all of the above, the antibody comprises a heavy chain variable (VH) region and a light chain variable (VL) region; wherein the VH region comprises an HCDR1 comprising the amino acid sequence of DYYIH (SEQ ID NO:31), an HCDR2 comprising the amino acid sequence of RVNPDHGDSYYNQKFKD (SEQ ID NO:32), and an HCDR3 comprising the amino acid sequence of ARNYLFDHW (SEQ ID NO:33), and wherein the VL region comprises a LCDR1 comprising the amino acid sequence of KASQDVGTAVA (SEQ ID NO:34), a LCDR2 comprising the amino acid sequence of WASIRHT (SEQ ID NO:35), and a LCDR3 comprising the amino acid sequence of HQFATYT (SEQ ID NO:8). In some embodiments of all of the above, the antibody comprises a heavy chain variable (VH) region and a light chain variable (VL) region the antibody is a murine, chimeric, or humanized antibody. In some embodiments of all of the above, the antibody comprises a heavy chain variable (VH) region and a light chain variable (VL) region the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDHGD SYYNQKFKDKATITADKSTDTAYMELSSLRSEDTAVYFCARNYLFDIWGQGTLVTVSS (SEQ ID NO:9): and wherein the VL region comprises the amino acid sequence of DIQMTQSPSSVSASVGDRVTITCKASQDVGTAVAWYQQKPGKAPKLLIYWASIRHTGVP SRFSGSGSGTDFTLTISSLQPEDFATYYCHQFATYTFGGGTKVEIK (SEQ ID NO:10). In some embodiments of all of the above, the antibody is a human IgG antibody. In some embodiments of all of the above, the antibody is a human IgG1, IgG2, and IgG4 antibody. In some embodiments of all of the above, the amino acid sequence of the heavy chain of the antibody is SEQ ID NO:1, and the amino acid sequence of the light chain of the antibody is SEQ ID NO:2.

In some embodiments of all of the above, the antibody-drug conjugate is Disitamab vedotin or a biosimilar thereof. In some embodiments of all of the above, the average DAR (i.e., Drug-to-Antibody Ratio) value of the antibody-drug conjugate is any number from 2 to 7. In some embodiments of all of the above, the average DAR value is 4±0.5.

In some embodiments of all of the above, the immune checkpoint inhibitor is a PD-1 antibody. In some embodiments of all of the above, the PD-1 antibody is Toripalimab, Dostarlimab, Prolgolimab, Tislelizumab, Camrelizumab, Sintilimab, Cemiplimab, Pembrolizumab, Nivolumab, Penpulimab, Genolimzumab, Zimberelimab, or Balstilimab. In some embodiments of all of the above, the immune checkpoint inhibitor is a PD-L1 antibody. In some embodiments of all of the above, the immune checkpoint inhibitor is a PD-L1 antibody is Durvalumab, Avelumab, Atezolizumab, Envafolimab, or RC98.

In some embodiments of all of the above, the patient has previously received one or more prior treatments, such as chemotherapy drugs, targeted therapy, immunotherapy or endocrine therapy. In some embodiments of all of the above, the urothelial cancer patient is a patient with locally advanced urothelial cancer that cannot be surgically resected, a patient with locally advanced or metastatic urothelial cancer, a patient with HER2-positive urothelial cancer, a patient with HER2 positive locally advanced or metastatic urothelial cancer, or a urothelial cancer patient who cannot tolerate platinum-based chemotherapy. In some embodiments of all of the above, the urothelial cancer patient is a patient with unresectable locally advanced or metastatic urothelial carcinoma. In some embodiments of all of the above, the urothelial cancer patient is a patient who is ineligible for or has refused cisplatin based chemotherapy. In some embodiments of all of the above, the urothelial cancer patient is a patient who has progressed after chemotherapy. In some embodiments of all of the above, the urothelial cancer patient is a patient who has experienced disease progression within 12 months of completion of neoadjuvant or adjuvant cisplatin-based chemotherapy.

In some embodiments of all of the above, the medicament is administered intranasally, subcutaneously, intradermally, intramuscularly or intravenously. In some embodiments of all of the above, the ADC is administered at a dosage of 1.5 mg/kg or 2.0 mg/kg. In some embodiments of all of the above, the ADC is administered every 2 weeks or 14 days.

In some embodiments of all of the above, administration of the antibody-drug conjugate and immune checkpoint inhibitor to the urothelial cancer patient results in progression-free survival (PFS) of greater than 7.5 months.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 is a schematic diagram of the structure of monomethvl auristatin E

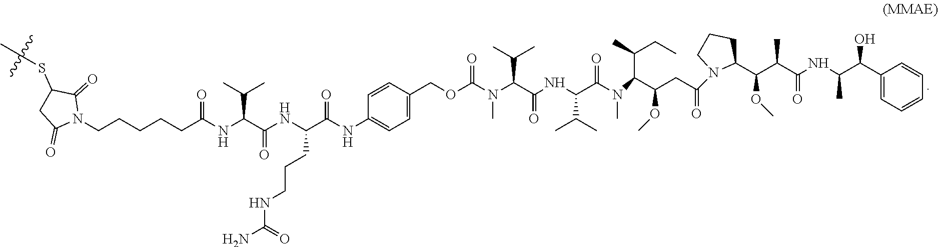 [SMILES: *SC1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)C1=O]

FIG. 2 is a schematic diagram of exemplary structures of the antibody-drug conjugates of the structural general formula Ab-(L-U)ₙ of the present disclosure under one potential set of conjugation conditions (L is linked to one or more interchain disulfide bond sites of the antibody through sulfhydryl conjugation) where n is 1, 2, 3, 4, 5, 6, 7, and 8, respectively; L is Maleimido-Caproyl-Valine-Citrulline-p-Aminobenzyloxy (mc-vc-pAB); U is MMAE (monomethyl auristatin E); and the structure of “-L-U” is as follows:

FIG. 3 is a schematic overview of the method used in a phase II clinical trial.

FIGS. 4A-D are graphical representations of patients' response to co-treatment with RC48-ADC and JS001. FIGS. 4A-C show patients' overall response rate, and FIG. 4D shows patients' progression free survival.

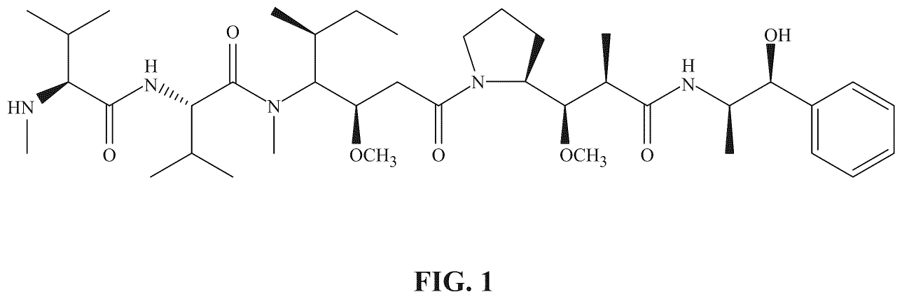

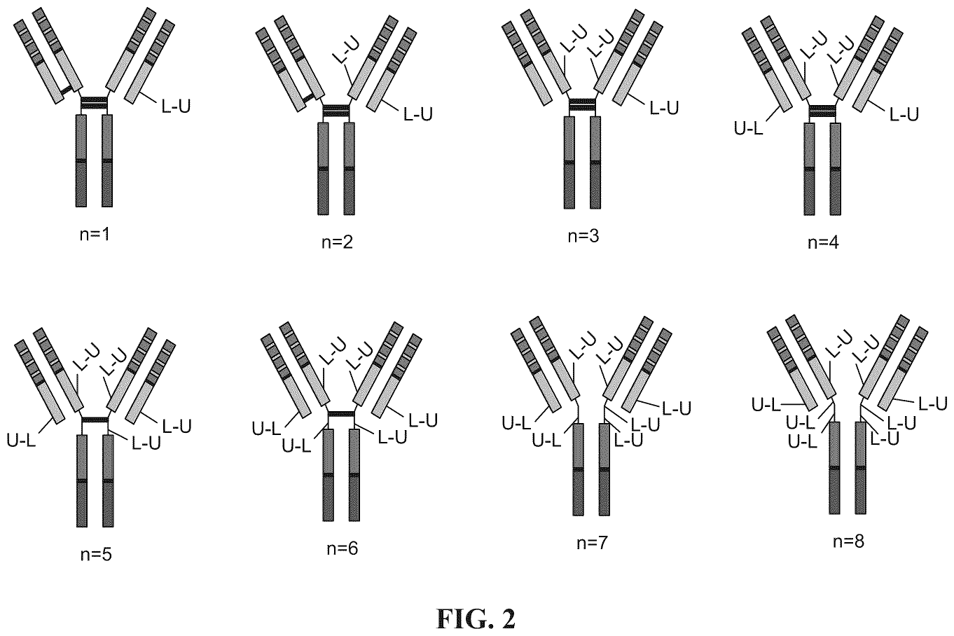

### DETAILED DESCRIPTION OF EMBODIMENTS

### I. Definitions

Unless otherwise defined, all technical and scientific terms used herein have the same meaning as understood by those of ordinary skill in the art. For definitions and terms in the field, professionals can refer to Current Protocols in Molecular Biology (Ausubel).

The three-letter and one-letter codes for amino acids used in the present disclosure are as described in J. biol. chem, 243, p 3558 (1968).

In the present disclosure, the determination or numbering method of the complementary determining regions (CDRs) of the variable domains of antibodies include the IMGT and Kabat systems, which are well known in the art.

The “antibody” used in the present disclosure encompasses a variety of antibody structures including, but not limited to, monoclonal antibodies, polyclonal antibodies, multispecific antibodies (e.g., bispecific antibodies), and antigen binding fragments. “Antigen binding fragment” used in the present disclosure refers to an antibody fragment comprising a heavy chain variable region or a light chain variable region of the antibody and being sufficient to retain the same binding specificity as its source antibody and sufficient affinity. In particular, the antigen binding fragments comprise Fab, F(ab′), and F(ab′)2, which contain at least one immunoglobulin fragment sufficient to make a specific antigen bind to the polypeptide. The above fragments can be prepared by synthesis, or by an enzymic method, or by chemical cutting of intact immunoglobulin, or genetically engineered by using recombinant DNA techniques. The production methods of the above fragments are well known in the art.

The term “murine antibody” as used in the present disclosure is a monoclonal antibody prepared according to the knowledge and skill in the art. During preparation, a corresponding antigen is injected into the test subjects, and then hybridomas expressing an antibody having the desired sequence or functional characteristics are isolated. In some embodiments, the murine antibody or antigen binding fragment thereof can further comprise a light chain constant region of murine κ or λ chain or a variant thereof, or further comprise a heavy chain constant region of murine IgG1, IgG2, IgG3, or a variant thereof.

The term “chimeric antibody” as used in the present disclosure is an antibody that is a fusion of a variable region of a murine antibody with a constant region of a human antibody, and can reduce immune response induced by the murine antibody. When establishing the chimeric antibody, hybridomas which secrete a murine specific monoclonal antibody are first established. Then, variable region genes are cloned from murine hybridoma cells, and as required, constant region genes are cloned from the human antibody. The mouse variable region genes and the human constant region genes are linked to form a chimeric gene and inserted into a human vector. Finally, chimeric antibody molecules are expressed in an eukaryotic industrial system or a prokaryotic industrial system. In some embodiments, the antibody light chain of the chimeric antibody further comprises a light chain constant region of human κ or λ chain or a variant thereof. The antibody heavy chain of the chimeric antibody can further comprise a heavy chain constant region of human IgG1, IgG2, IgG3. IgG4, or a variant thereof. The constant region of the human antibody can be selected from the heavy chain constant region of human IgG1, IgG2, IgG3, or IgG4, or a variant thereof, and comprise the heavy chain constant region of human IgG2 or IgG4. Alternatively, IgG4 which has no ADCC toxicity (antibody-dependent cell-mediated cytotoxicity) after an amino acid mutation occurred is used.

The term “humanized antibody” as used in the present disclosure, also known as CDR-grafted antibody, refers to a antibody generated by grafting of a mouse CDR sequence into human antibody variable region framework (i.e., human germline antibody framework sequences of different types). It comprises a CDR region derived from a non-human antibody and the rest of the antibody molecule is derived from one human antibody (or several human antibodies). Furthermore, in order to preserve binding affinity, some residues of the framework region (known as FR) segments can be modified (Jones et al., Nature, 321:522-525, 1986: Verhoeyen et al., Science, 239.1534-1536, 1988: and Riechmann et al. Nature, 332:323-327, 1988). The humanized antibodies or fragments thereof according to the present disclosure can be prepared by techniques known to those skilled in the art (e.g., as described in Singer et al., J. Immun. 150: 2844-2857, 1992; Mountain et al., Biotechnol. Genet. Eng. Rev., 10: 1-142, 1992; or Bebbington et al., Bio/Technology, 10: 169-175, 1992).

The term average DAR” value as used in the present disclosure, namely the Drug-to-Antibody Ratio, refers to the average value of the number of drugs linked to the antibody in the antibody-drug conjugate preparation.

The term “sulfhydryl conjugation” as used in the present disclosure refers to a conjugation means by which the linker is covalently linked to a free sulfhydryl group on the antibody. Cysteine exists in the form of a disulfide bond in the antibody, and there are 4 pairs of interchain disulfide bonds in an IgG antibody, which are easily reduced. Therefore, during the preparation of the antibody-drug conjugate, the 4 pairs of interchain disulfide bonds in the IgG antibody are frequently reduced, which produces the above-mentioned “free sulfhydryl group on the antibody”. Moreover, since there are 4 pairs of interchain disulfide bonds in the IgG antibody, when they are reduced, a maximum of 8 free sulfhydryl groups will be generated. An IgG antibody will therefore have a maximum of 8 sulfhydryl conjugation sites. Thus, when in the antibody-drug conjugate of the general formula Ab-(L-U)ₙ n is 1, “L-U” can be covalently linked to any 1 site of the 8 sulfhydryl conjugation sites; similarly, when n is 2, “L-U” can be covalently linked to any 2 sites of the 8 sulfhydryl conjugation sites; when n is 3, “L-U” can be linked to any 3 sites of the 8 sulfhydryl conjugation sites; when n is 4, “L-U” can be covalently linked to any 4 sites of the 8 sulfhydryl conjugation sites; when n is 5, “L-U” can be covalently linked to any 5 sites of the 8 sulfhydryl conjugation sites: when n is 6, “L-U” can be covalently linked to any 6 sites of the 8 sulfhydryl conjugation sites; when n is 7, “L-U” can be covalently linked to any 7 sites of the 8 sulfhydryl conjugation sites; and when n is 8, ‘L-U’ can be covalently linked to the 8 sulfhydryl conjugation sites.

### II. Uses and Methods

Certain aspects of the present disclosure relate to antibody-drug conjugates that bind HER2 (as well as methods and uses for the same). In some embodiments, the antibody-drug conjugate involved has the structure of the general formula Ab-(L-U)n, where Ab represents anti-HER2 (Human Epidermal Growth Factor Receptor 2) antibody: L represents a linker; U represents conjugated cytotoxic molecules; and n is an integer from 1 to 8 (e.g., 1, 2, 3, 4, 5, 6, 7, 8), and represents the number of cytotoxic molecules bound to each antibody.

In some embodiments, the cytotoxic molecule is an auristatin, or an analog or derivative thereof. Auristatins are derivatives of the natural product dolastatin. Exemplary auristatins include dolostatin-10, auristatin E, auristatin T, MMAE (N-methylvaline-valine-dolaisoleuine-dolaproine-norephedrine or monomethyl auristatin E) and MMAF (N-methylvaline-valine-dolaisoleuine-dolaproine-phenylalanine or dovaline-valine-dolaisoleunine-dolaproine-phenylalanine), AEB (ester produced by reacting auristatin E with paraacetyl benzoic acid), AEVB (ester produced by reacting auristatin E with benzovlvaleric acid), and AFP (dimethylvaline-valine-dolaisoleuine-dolaproine-phenylalanine-p-phenylenediamine or auristatin phenylalanine phenylenediamine). WO 2015/057699 describes PEGylated auristatins including MMAE. Additional dolostatin derivatives contemplated for use are disclosed in U.S. Pat. No. 9,345,785, incorporated herein by reference for any purpose.

In some embodiments, the cytotoxic molecule is MMAE. In other embodiments, the cytotoxic agent is MMAF.

In some embodiments, the anti-HER2 (Human Epidermal Growth Factor Receptor 2) antibody or the functional fragment thereof in the antibody-drug conjugate provided by the present disclosure comprises a heavy chain variable region and a light chain variable region, where the CDR of the heavy chain variable region and/or the CDR of the light chain variable region have the same CDR sequences as Disitamab vedotin: the linker L comprises Maleimido-Caproyl-Valine-Citrulline-p-Aminobenzyloxy (mc-vc-pAB): and the cytotoxic molecules U comprise MMAE (monomethyl auristatin E).

In some embodiments, the linker L is covalently Inked to the antibody by means of sulfhydryl conjugation, and the linking site is the interchain disulfide bond site of the antibody.

In some preferred examples, the antibody-drug conjugates of the present disclosure is a mixture of antibody-drug conjugates linked with 2-7 cytotoxic molecules, where the average DAR (i.e., Drug-to-Antibody Ratio) value of the antibody-drug conjugates is any number from 2 to 7: more preferably, the average DAR value of the antibody-drug conjugates of the present disclosure is approximately equal to 2, 3, 4, 5, 6, or 7. In some specific examples of the present disclosure, the average DAR value of the antibody-drug conjugates of the present disclosure is 4 t 0.5.

More specifically, the antibody-drug conjugate of the present disclosure is Disitamab vedotin, which is an antibody-drug conjugate targeting a HER2 target, where the linker moiety L is Maleimido-Caproyl-Valine-Citrulline-p-Aminobenzyloxy (mc-vc-pAB): the cytotoxic molecules U comprise MMAE (monomethyl auristatin E): the linker L is covalently linked to the antibody by means of sulfhydryl conjugation; and the average DAR value is 4±0.5.

In some embodiments, the corresponding CDRs 1-3 of the heavy chain variable region and the light chain variable region of the anti-HER2 antibody involved in the present disclosure are as follows (IMGT numbering):

TABLE 1
IMGT

| | | |
|---|---|---|
| HCDR1: | GYTFTDYY | SEQ ID NO: 3 |
| HCDR2: | VNPDHGDS | SEQ ID NO: 4 |
| HCDR3: | ARNYLFDH | SEQ ID NO: 5 |
| LCDR1: | QDVGTA | SEQ ID NO: 6 |
| LCDR2: | WAS | SEQ ID NO: 7 |
| LCDR3: | HQFATYT | SEQ ID NO: 8 |

TABLE 2
Kabat

| | | |
|---|---|---|
| HCDR1: | DYYIH | SEQ ID NO: 31 |
| HCDR2: | RVNPDHGDSYYNQKFKD | SEQ ID NO: 32 |
| HCDR3: | ARNYLFDHW | SEQ ID NO: 33 |
| LCDR1: | KASQDVGTAVA | SEQ ID NO: 34 |
| LCDR2: | WASIRHT | SEQ ID NO: 35 |
| LCDR3: | HQFATYT | SEQ ID NO: 8 |

In some embodiments, the anti-HER2 antibody comprises the corresponding CDRs 1-3 of the heavy chain variable regions and the light chain variable region represented by SEQ ID Nos:3-8, but with 1, 2, or 3 substitutions (e.g., conservative substitutions), insertions, or deletions relative to SEQ ID Nos:3-8, but an anti-HER2 antibody comprising that sequence retains the ability to bind to HER2. In some embodiments, the anti-HER2 antibody comprises the corresponding CDRs 1-3 of the heavy chain variable regions and the light chain variable region represented by SEQ ID Nos: 31-35 and 8, but with 1, 2, or 3 substitutions (e.g., conservative substitutions), insertions, or deletions relative to SEQ ID Nos: 31-35 and 8, but an anti-HER2 antibody comprising that sequence retains the ability to bind to HER2.

In some embodiments, the anti-HER2 (Human Epidermal Growth Factor Receptor 2) antibody in the antibody-drug conjugate provided by the present disclosure is murine, chimeric, humanized or fully human, preferably a humanized monoclonal antibody. In some embodiments, the antibody is a monoclonal antibody.

In some embodiments, the anti-HER2 (Human Epidermal Growth Factor Receptor 2) antibody in the antibody-drug conjugate provided by the present disclosure is IgG, including IgG1, IgG2, IgG3, and IgG4, and more preferably IgG1, IgG2, and IgG4.

In some embodiments, the anti-HER2 antibody comprises a heavy chain variable (VH) region and a light chain variable (VL) region; wherein the VH region comprises an amino acid sequence with at least 90%, at least 91%, at least 92%, at least 93%, at least 94%, at least 95%, at least 96%, at least 97%, at least 98%, at least 99%, or 100% identity to the sequence EVQLVQSGAEVKKPGATVKISCKVSGYTFTDYYIHWVQQAPGKGLEWMGRVNPDHGD SYYNQKFKDKATITADKSTDTAYMELSSLRSEDTAVYFCARNYLFDHWGQGTLVTVSS (SEQ ID NO:9); and/or wherein the VL region comprises an amino acid sequence with at least 90%, at least 91%, at least 92%, at least 93%, at least 94%, at least 95%, at least 96%, at least 97%, at least 98%, at least 99%, or 100% identity to the sequence DIQMTQSPSSVSASVGDRVTITCKASQDVGTAVAWYQQKPGKAPKLLIYWASIRHTGVP SRFSGSGSGTDFTLTISSLQPEDFATYYCHQFATYTFGGGTKVEIK (SEQ ID NO:10). In certain embodiments, the VH sequence (e.g., having at least 90%, 91%, 92%, 93%, 94%, 95%, 96%, 97%, 98%, or 99% identity to SEQ ID NO:9) contains substitutions (e.g., conservative substitutions), insertions, or deletions relative to SEQ ID NO:9, but an anti-HER2 antibody comprising that sequence retains the ability to bind to HER2. In certain embodiments, a total of 1 to 10 amino acids have been substituted, inserted and/or deleted in SEQ ID NO: 9. In certain embodiments, substitutions, insertions, or deletions occur in regions outside the CDRs (i.e., in the FRs). In certain embodiments, the VL sequence (e.g., having at least 90%, 91%, 92%, 93%, 94%, 95%, 96%, 97%, 98%, or 99% identity to SEQ ID NO:10) contains substitutions (e.g., conservative substitutions), insertions, or deletions relative to SEQ ID NO:10, but an anti-HER2 antibody comprising that sequence retains the ability to bind to HER2. In certain embodiments, a total of 1 to 10 amino acids have been substituted, inserted and/or deleted in SEQ ID NO: 10. In certain embodiments, substitutions, insertions, or deletions occur in regions outside the CDRs (i.e., in the FRs).

In some embodiments, the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDHGD SYYNQKFKDKATITADKSTDTAYMELSSLRSEDTAVYFCARNYLFDHWGQGTLVTVSS (SEQ ID NO:9): and wherein the VL region comprises the amino acid sequence of DIQMTQSPSSVSASVGDRVTITCKASQDVGTAVAWYQQKPGKAPKLLIYWASIRHTGVP SRFSGSGSGTDFTLTISSLQPEDFATYYCHQFATYTFGGGTKVEIK (SEQ ID NO:10).

In some embodiments, the heavy chain amino acid sequence of the antibody Ab in the antibody-drug conjugate involved in the present disclosure is shown in SEQ ID NO: 1, and the light chain amino acid sequence thereof is shown in SEQ ID NO: 2. In some embodiments, the heavy chain comprises the amino acid sequence of SEQ ID NO:1 without the C-terminal lysine.

| Heavy chain amino acid sequence | |
|---|---|
| SEQ ID NO: 1 | |
| EVQLVQSGAE VKKPGATVKI SCKVSGYTFT DYYIHWVQQA PGKGLEWMGR | 50 |
| VNPDHGDSYY NQKFKDKATI TADKSTDTAY MELSSLRSED TAVYFCARNY | 100 |
| LFDHWGQGTL VTVSSASTKG PSVFPLAPSS KSTSGGTAAL GCLVKDYFPE | 150 |
| PVTVSWNSGA LTSGVHTFPA VLQSSGLYSL SSVVTVPSSS LGTQTYICNV | 200 |
| NHKPSNTKVD KKVEPKSCDK THTCPPCPAP ELLGGPSVFL FPPKPKDTLM | 250 |
| ISRTPEVTCV VVDVSHEDPE VKFNWYVDGV EVHNAKTKPR EEQYNSTYRV | 300 |
| VSVLTVLHQD WLNGKEYKCK VSNKALPAPI EKTISKAKGQ PREPQVYTLP | 350 |
| PSREEMTKNQ VSLTCLVKGF YPSDIAVEWE SNGQPENNYK TTPPVLDSDG | 400 |
| SFFLYSKLTV DKSRWQQGNV FSCSVMHEAL HNHYTQKSLS LSPGK | 445 |
| Light chain amino acid sequence | |
| SEQ ID NO: 2 | |
| DIQMTQSPSS VSASVGDRVT ITCKASQDVG TAVAWYQQKP GKAPKLLIYW | 50 |
| ASIRHTGVPS RFSGSGSGTD FTLTISSLQP EDFATYYCHQ FATYTFGGGT | 100 |
| KVEIKRTVAA PSVFIFPPSD EQLKSGTASV VCLLNNFYPR EAKVQWKVDN | 150 |
| ALQSGNSQES VTEQDSKDST YSLSSTLTLS KADYEKHKVY ACEVTHQGLS | 200 |
| SPVTKSFNRG EC | 212 |

Certain aspects of the uses and methods relate to immune checkpoint inhibitors. Exemplary PD-1 antibodies include Toripalimab, Dostarlimab, Prolgolimab, Tislelizumab, Camrelizumab, Sintilimab, Cemiplimab, Pembrolizumab, Nivolumab, Penpulimab. Genolimzumab, Zimberelimab, and Balstilimab. Exemplary PD-L1 antibodies include Durvalumab. Avelumab, Atezolizumab, and Envafolimab.

The antibody-drug conjugate and immune checkpoint inhibitor may be administered in any order. For example, an antibody-drug conjugate and immune checkpoint inhibitor may be administered sequentially (at different times) or concurrently (at the same time). In some embodiments, an antibody-drug conjugate and immune checkpoint inhibitor are in separate compositions. In some embodiments, an antibody-drug conjugate and immune checkpoint inhibitor are in the same composition.

In some embodiments, the patient of the present disclosure has previously received one or more prior treatments, such as chemotherapy drugs, targeted therapy, immunotherapy and endocrine therapy.

In some embodiments, the patient is one with locally advanced urothelial cancer that cannot be surgically resected, with locally advanced or metastatic urothelial cancer, with HER2-positive urothelial cancer, with HER2 positive locally advanced or metastatic urothelial cancer, or one who cannot tolerate platinum-based chemotherapy.

In some embodiments, the patient of the present disclosure is a patient whose chemotherapy has failed.

### EXAMPLES

The examples below are but it is not intended to limit the scope of the present disclosure. The experimental methods not specified for the specific conditions in the following examples are selected according to conventional methods and conditions, or according to the product instructions.

### Example 1 Synergistic Therapeutic Effect of Disitamab Vedotin (RC48) in Combination with PD-L1 Antibody

The PD-L1 antibody (RC98) used was disclosed in WO2021/037007A1. The heavy chain amino acid sequence is depicted in SEQ ID NO: 21, and the light chain amino acid sequence is depicted in SEQ ID NO: 22. The CDR1-3 sequences of the heavy chain of the antibody are depicted in SEQ ID Nos: 23-25, the CDR1-3 sequences of the light chain of the antibody are depicted in SEQ ID Nos: 26-28, the variable region of the heavy chain amino acid sequence is depicted in SEQ ID NO: 29, and the variable region of the light chain amino acid sequence is depicted in SEQ ID NO: 30.

It was evaluated whether there was a synergistic inhibitory effect on the growth of the subcutaneously transplanted tumors from human colon cancer cells I-IT-29 (source: ATCC) of NSG mice (source: Biocytogen jiangsu Co., Ltd.) implanted with human PBMCs.

Method: 2×10⁶ human colon cancer cells HT-29 were inoculated into the right armpit near the back of the NSG mice. When the tumor volume grew to about 100-300 mm³, 5×10⁶ human PBMCs were intravenously implanted in each NSG mouse. On the next day, the mice were randomly divided into 4 groups according to tumor volume, named as Control (saline) group, RC98 (10 mg/kg) group, RC48-ADC (2 mg/kg) group, and RC98 (10 mg/kg) & RC48-ADC (2 mg/kg) combined administration group, with 5 experimental animals in each group. Sodium chloride injection was administrated intravenously to the mice from the Control (saline) group once a week for a total of 2 times; the mice from the RC98 group w % ere dosed intraperitoneally twice a week for a total of 8 times; the mice from the RC48-ADC group were dosed intravenously once a week for a total of 2 doses; and for the RC98 & RC48-ADC combined administration group. RC98 was administered intraperitoneally to the mice twice a week for a total of 8 times, and RC48-ADC was administered intravenously once a week for a total of 2 times. It was evaluated whether the combination of RC48-ADC & RC98 had a synergistic inhibitory effect on the growth of HT-29 subcutaneously transplanted tumors, based on the formula for calculating whether the two drugs have a synergistic effect:

TABLE 3
Changes in tumor volume in tumor-bearing mice (Mean ± SEM)

| | | | | | | | | Using TGI_{RTV} | |
|---|---|---|---|---|---|---|---|---|---|
| | | | | | | | | (%) | Using TGI_{TW} |
| | | | | | | | | as the effect | as the effect |
| | | | | | | Tumor | | index to | index to |
| | Dosage | TV (mm³) | | T/C | TGI_{RTV} | weight | TGI_{TW} | calculate | calculate |
| Group | (mg/kg) | D 0 | D 29 | (%) | (%) | (g) | (%) | the Q value | the Q value |
| Control | — | 254.4 ± | 1846.7 ± | — | | 2.08 ± | — | 1.5 | 2.0 |
| (saline) | | 21.9 | 109.8 | | | 0.12 | | | |
| RC98 | 10 | 253.2 ± | 1697.5 ± | 91 | 9 | 2.03 ± | 3 | | |
| | | 20.9 | 146.2 | | | 0.19 | | | |
| RC48- | 2 | 257.3 ± | 1577.7 ± | 85 | 15 | 1.70 ± | 18 | | |
| ADC | | 21.1 | 121.9 | | | 0.15 | | | |
| RC98& | 10&2 | 259.0 ± | 1214.4 ± | 65 | 35 | 1.22 ± | 41 | | |
| RC48- | | 23.6 | 127.3** | | | 0.13**^{ΔΔ#} | | | |

Note:
compared with the Control group,
**P < 0.01; compared with the RC98 group,
^{ΔΔ}<0.01; and compared with RC48-ADC,
^{#}P < 0.05.

Results: (1) During the trial, no drug-related weight loss was found in each group of animals. (2) The results of tumor volume measurement showed that there was no statistical difference between the RC98 group or the RC48-ADC group and the Control group (P>0.05), and the statistical difference between the combined administration group and the Control group was extremely significant (P<0.01). (3) The results of the tumor mass detection showed that there was no statistical difference between the RC98 group or the RC48-ADC group and the Control group (P>0.05), and the tumor inhibition rates (TGITW %) were 3% and 18%, respectively. The statistical difference between the combined administration group and the Control group was extremely significant (P<0.01), and the tumor inhibition rate (TGITW %) was 41%. (4) Based on the formula for calculating whether there was a synergistic effect Q value between the two drugs: Q=P0/[P(A)+P(B)−P(A)P(B)], Q=1.5 was calculated by using the relative tumor inhibition rate (TGIRTV) as the effect index, or Q=2.0 was calculated by using the tumor inhibition rate (TGITW) as the effect index.

Conclusion: The combination of RC98 and RC48-ADC had a significant inhibitory effect on the subcutaneously transplanted tumors from the human colon cancer cells HT-29 of the NSG mice implanted with human PBMCs. The Q values calculated by using the two different effect indexes were both greater than 1.2. It can be concluded that the combination of RC98 and RC48-ADC has a synergistic effect on the proliferation inhibition of HT-29 subcutaneously transplanted tumors.

### Example 2 Clinical Trial of Treatment with Disitamab Vedotin (RC48) in Combination with PD-1 Antibody (Toripalimab, JS001): Interim Analysis

RC48 in combination with an anti-PD-1 monoclonal antibody (Toripalimab, JS001) was used to treat mUC, including patients who could not tolerate platinum-based chemotherapy in a first-line treatment.

The heavy chain amino acid sequence of Toripalimab was shown in SEQ ID NO: 11, and the light chain amino acid sequence thereof is depicted in SEQ ID NO: 12. The CDR1-3 sequences of the heavy chain of the antibody are depicted in SEQ ID NOs: 13-15, the CDR1-3 sequences of the light chain of the antibody are depicted in SEQ ID NO: 16-18, the variable region of the heavy chain amino acid sequence is depicted in SEQ ID NO: 19, and the variable region of the light chain amino acid sequence is depicted in SEQ ID NO: 20.

### Key Inclusion Criteria:

urothelial cancer confirmed histologically to be unresectable, locally advanced, or metastatic;
slow progression and inability to tolerate cisplatin-based chemotherapy after the treatment with at least 1 prior systemic chemotherapy regimen, within 12 months after completion of neoadjuvant or cisplatin-based adjuvant chemotherapy; and
ECOG performance status of 0-1.

### Dosing Regimen:

RC48 2.0 mg/kg+JS001 3 mg/kg Q2W (n=3)
RC48 2.0 mg/kg+JS001 3 mg/kg Q2W (n=13)
RC48 1.5 mg/kg+JS001 3 mg/kg Q2W (n=3)

TABLE 4
Data of patients (%)

| Characteristics | Total (N = 19) | |
|---|---|---|
| Age (years, median, range) | 66 | (52, 76) |
| Male (n, %) | 12 | (63.2%) |
| ECOG PS status (n, %) | | |
| 0 | 4 | (21.0%) |
| 1 | 15 | (79.0%) |
| HER2 Expression | | |
| IHC 3+ (n, %) | 3 | (15.8%) |
| IHC 2+ (n, %) | 9 | (47.4%) |
| IHC 1+ (n, %) | 5 | (26.3%) |
| IHC 0 (n, %) | 2 | (10.5%) |
| Primary Lesion | | |
| Bladder (n, %) | 6 | (31.6%) |
| Renal pelvis (n, %) | 5 | (26.3%) |
| Ureter (n, %) | 5 | (26.3%) |
| Multifocal (n %) | 3 | (15.8%) |
| Visceral metastases (n, %) | 14 | (73.7%) |
| Lung (n, %) | 6 | (31.6%) |
| Liver (n, %) | 8 | (42.1%) |
| Prior systemic treatment | | |
| 0 Line (n, %) | 10 | (52.6%) |
| ≥1 Lines (n, %) | 9 | (47.4%) |
| PD-L1 status by combined positive score (CPS, n, %) | | |
| <10 | 12 | (63.2%) |
| ≥10 | 7 | (36.8%) |

Treatment Outcomes: A total of 19 patients completed at least one treatment dose, and 17 patients completed at least one efficacy evaluation. The results showed that the objective remission rate (ORR) was 94.1% (16/17), of which 3 patients achieved complete remission and 13 achieved partial remission. Among the 19 patients who received study treatment, the most common TRAEs reported were anorexia (15, 79.0%), fatigue (13, 68.4%), elevated ALT/AST (11, 57.9%) and peripheral sensory neuropathy (11, 57.9%). In Her2-expressing patients (Her2 1+, 2+, or 3+), the ORR reached 100%

### Example 3 Clinical Trial of Treatment with Disitamab Vedotin (RC48) in Combination with PD-1 Antibody (Toripalimab, JS001)

This example includes further details regarding the clinical trial described in Example 2, as well as analysis of the data after enrollment of additional participants.

The prognosis of metastatic urothelial carcinoma is poor, and 5-year survival rate of locally advanced or metastatic urothelial carcinoma (la/mUC) is about 15%. There is still an unmet clinical need for patients who are intolerant to first-line cisplatin chemotherapy treatment or who fail platinum therapy. Break-through treatments such as antibody drug conjugates (ADC) and immune checkpoint inhibitors (ICI) as monotherapy have achieved promising efficacy results, although the need for novel treatments or effective combination therapies remains unmet.

Preclinical studies showed that ADC linked to MMAE (monomethyl auristatin E) elicited immunogenic cell death (ICD) and had a direct effect on dendritic cell (DC) maturation and activation, which may have enhanced antitumor immunity. Recent phase II studies assessed two recombinant humanized anti-HER2 monoclonal antibody-MMAEs compounds (hereinafter referred to collectively as “RC48-ADC”) in the treatment of HER2-positive locally advanced or metastatic uroepithelial carcinoma, which showed positive efficacy and safety results. In the NCT03507166 study, in which RC48-ADC was the second-line treatment of HER2-overexpressed (IHC 2+/3+) la/mUC, the cORR was 51.2%, the mPFS was 6.9 months, and the mOS was 13.9 months. In the NCT03809013 study assessing a second RC48-ADC compound, in which RC48-ADC was the second-line treatment of HER2-overexpressed (IHC 2+/3+) la/mUC, the cORR was 50.0%, the mPFS was 5.1 months, and the mOS was 14.2 months. Following these studies, RC48-ADC compounds were recognized as a breakthrough therapy by FDA and CDE.

A separate clinical trial assessing efficacy and safety of the anti-PD-1 monoclonal antibody known as toripalimab (JS001) as the second-line treatment of la/mUC, the POLARIS-03 study (NCT03113266), demonstrated a cORR that was 26%, mPFS that was 2.3 months, and mOS that was 14.4 months.

The study described in this example was designed to establish the clinical relevancy of the described RC48-ADC and JS001 combination therapy model, in particular to evaluate the safety and pharmacokinetics of RC48-ADC and JS001 combination therapy in patients with advanced or metastatic urothelial cancer.

### Method

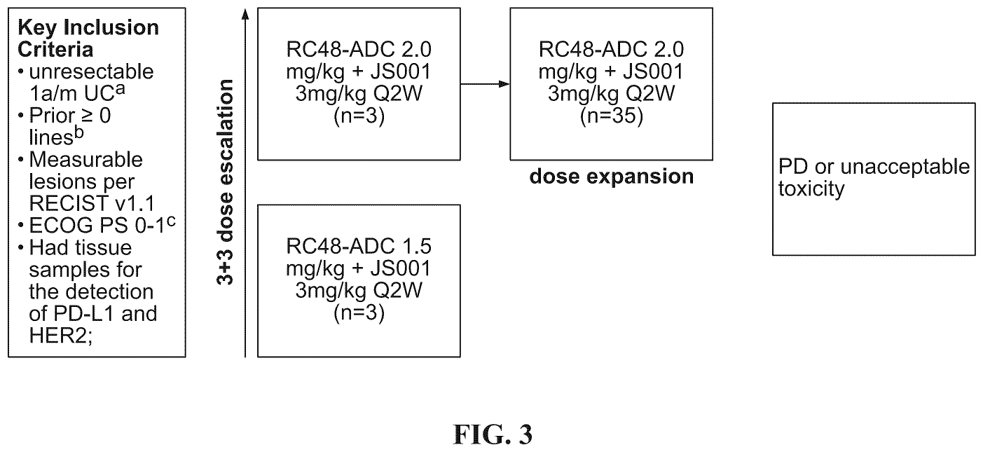

The RC48-ADC compound was administered in combination with JS001 to n=3 patients as a biweekly (Q2W) injection at 3.0 mg/kg JS001 and either 1.5 mg/kg or 2.0 mg/kg RC48-ADC in a 3+3 dose escalation phase II clinical trial to assess for any initial safety concerns. The recommended phase II dose (RP2D) of RC48-ADC at 2 mg/kg was determined by the initial 6-patient safety lead-in of RC48-ADC in combination with the standard approved JS001 dose 3 mg/kg. A total of n=35 patients then received in combination RC48 at 2.0 mg/kg and JS001 at 3 mg/kg Q2W in the expansion stage, for a total of n=41 enrolled patients. Patient characteristics are reported below in Table 5. Patients were monitored to ensure no dose limiting toxicities (DLT) arose. Patients were followed for up to 12 months for most endpoints and for 60 weeks to assess objective response rate (ORR) in order to analyze the co-treatment safety and efficacy profiles as described below. FIG. 3 depicts an outline of the study method used in this clinical trial.

Inclusion criteria included:
(a) Age (≥18 years old);
(b) Sex (all);
(c) Patients with locally advanced or metastatic malignant urothelial carcinoma that was platinum naïve and cisplatin ineligible or had progressed after at least one line standard systemic chemotherapy (including progression within 12 months of neo-/adjuvant therapy)
(d) Eastern Cooperative Oncology Group (ECOG) performance status (PS) 0 or 1;
(e) Demonstration of adequate organ function as defined by the following criteria:
   (i) absolute neutrophil count (ANC) ≥1.0 times the upper limit of normal (ULN) or CrCl <60 mL/min;
   (ii) platelets ≥100 times 10⁹/L;
   (iii) total serum creatinine ≤1.5 times the ULN;
   (iv) serum aspartate transaminase (AST) and serum alanine transaminase (ALT) ≤2.5 times the ULN, or AST and ALT ≤5 times the ULN if liver function abnormalities were due to underlying malignancy: normal serum creatinine;
   (v) left ventricular ejection fraction (LVEF) ≥50%; and
   (vi) hemoglobin ≥9 g/dL.

Exclusion criteria included:
(a) allergy to RC48-ADC or JS001 or their components;
(b) received anti-cancer therapy including chemotherapy, radiotherapy, immunotherapy, or other clinical trial treatments within 3 weeks of starting study treatment;
(c) unresolved toxicities from prior anti-cancer therapy, except for alopecia;
(d) previously treated with HER2-ADC and/or anti-PD-1 or anti-PD-L1 or anti-PD-L2 therapies;
(e) underwent major surgery within 4 weeks of first dose of study drug and not completely recovered;
(f) received a vaccine within 4 weeks of first dose of study drug;
(g) clinically significant cardiovascular disease active at study start date or within the previous 6 months;
(h) history of other neoplastic disease within 3 years prior to the receiving the study drug, with the exception of resolved/curable cancers such as basal skin cancer or squamous cell skin cancer;
(i) metastasis to the central nervous system (CNS) and/or carcinomatous meningitis, with the exception of patients who received treatment of metastasis or CNS and/or carcinomatous meningitis and had stable disease for at least 3 months and no evidence of progression within 4 weeks of first dose of study treatment;
(j) evidence of new or expanded metastasis;
(k) treated with radiotherapy, surgery, or steroid therapy within 4 weeks of the first dose of study treatment;
(l) history of allogeneic hematopoietic stem cell transplantation or organ transplantation;
(m) receipt of systemic steroid therapy within the previous 2 years before the first dose of study treatment;
(n) testing HIV positive;
(o) active hepatitis B or C virus (HBV or HCV) or tuberculosis infection;
(p) positive for other disorders deemed of clinical significance according to the researcher's judgment; and
(q) unwilling or unable to participate in all required study evaluations and procedures.

The primary outcome measure included an analysis of safety and tolerability of RC48-ADC and JS001 combination therapy in order to identify the recommended dose for patients. This outcome measure included the assessment of dose limiting toxicities (DLTs) and adverse events (AEs).

Secondary outcome measures included the following:
(a) objective response rate (ORR);
(b) progression free survival (PFS);
(c) overall survival (OS); and
(d) characterization of pharmacokinetics (PK).

TABLE S
Demographics and baseline characteristics
of clinical trial-enrolled patients.

| Characteristics | N = 41 patients | |
|---|---|---|
| Age (years, median, range) | 66.0 | (42, 76) |
| Male (n, %) | 22 | (53.66%) |
| ECOG PS status (n, %) | | |
| 0 | 12 | (29.27%) |
| 1 | 29 | (70.73%) |
| Primary Lesion (n, %) | | |
| Bladder | 10 | (24.39%) |
| Renal pelvis | 12 | (29.27%) |
| Ureter | 10 | (24.39%) |
| Urethra | 3 | (7.32%) |
| Multiple Primary | 6 | (14.63%) |
| Metastatic Lesion (n, %) | | |
| Visceral | 22 | (53.66%) |
| Lung | 17 | (41.46%) |
| Liver | 10 | (24.39%) |
| Bone | 9 | (21.95%) |
| Prior systemic treatment (n, %) | | |
| 0 Line | 25 | (60.98%) |
| ≥1 Lines | 16 | (39.02%) |
| HER2 Expression (n, %) | | |
| IHC 3+ | 5 | (12.20%) |
| IHC 2+ | 19 | (46.34%) |
| FISH− | 16 | (39.02%) |
| FISH+ | 3 | (7.32%) |
| IHC 1+ | 14 | (34.15%) |
| IHC 0 | 3 | (7.32%) |
| PD-L1 Status (n, %) | | |
| − | 28 | (68.29%) |
| + | 13 | (31.71%) |
| HER2 & PD-L1 Expression (n, %) | | |
| HER2 IHC (2+/3+), PD-L1(+) | 8 | (19.51%) |
| HER2 IHC (2+/3+), PD-L1(−) | 16 | (39.02%) |
| HER2 IHC (1+), PD-L1(+) | 4 | (9.76%) |
| HER2 IHC (1+), PD-L1(−) | 10 | (24.39%) |
| HER2 IHC (0), PD-L1(+) | 1 | (2.44%) |
| HER2 IHC (0), PD-L1(−) | 2 | (4.88%) |

### Safety

RC48-ADC and JS001 combination therapy was shown to be well-tolerated with promising efficacy in patients with la/mUC. Indeed, most of the treatment-related adverse events (TRAEs) were grade 1-2, most commonly arising as anorexia and hypertriglyceridemia. Tables 6-7 provide the safety results from this study. Among the 41 patients receiving study treatment, the most commonly reported TRAEs were increases in AST (65.9%) and ALT (63.4%), peripheral sensory neuropathy (63.4%), asthenia (58.5%), decrease in appetite (56.1%), hypertriglyceridemia (56.1%), and increase in γ-glutamyltransferase (51.2%). The reported TRAEs that were Grade ≥3 included increases in γ-glutamyltransferase (12.2%) and ALT (7.3%), asthenia (7.3%), and hypertriglyceridemia (7.3%). Immune-related adverse events (irAEs) were reported in 16 patients, including immune-related pneumonitis, interstitial lung disease, hepatitis, myositis, hyperglycemia, and rash.

TABLE 6
Treatment-related adverse events (TRAEs) results for
patients receiving RC48-ADC and JS001 combination
therapy, with incidence ≥20% and Grade ≥3 TRAEs.

| | All grades - | | Grade ≥3 - | |
|---|---|---|---|---|
| TRAE | n (%) | | n (%) | |
| Overall | 41 | (100%) | 15 | (%) |
| Aspartate aminotransferase increase | 27 | (65.85%) | 2 | (%) |
| Alanine aminotransferase increase | 26 | (63.41%) | 3 | (%) |
| Peripheral sensory neuropathy | 26 | (63.41%) | 0 | |
| Asthenia | 24 | (58.54%) | 3 | (%) |
| Appetite decrease | 23 | (56.1%) | 0 | |
| Hypertriglyceridemia | 23 | (56.1%) | 3 | (%) |
| γ-glutamyltransferase increase | 21 | (51.22%) | 5 | (%) |
| Alopecia | 17 | (41.46%) | 0 | |
| Nausea | 16 | (39.02%) | 0 | |
| Hypercholesterolemia | 15 | (36.59%) | 0 | |
| White blood cell count decrease | 14 | (34.15%) | 1 | (%) |
| Blood creatine phosphokinase increase | 14 | (34.15%) | 1 | (%) |

| | | | |
|---|---|---|---|
| Anemia | 14 | (34.15%) | 0 |

TABLE 7
Immune-related adverse events (irAE) results for patients
receiving RC48-ADC and JS001 combination therapy.

| irAE | All Grades - n (%) | |
|---|---|---|
| Overall | 16 | (39.02%) |
| Interstitial lung disease | 3 | (7.32%) |
| Immune-related pneumonitis | 5 | (12.2%) |
| Rash | 8 | (19.51%) |
| Hyperglycemia | 1 | (2.44%) |
| Immune-related hepatitis | 1 | (2.44%) |
| Immune-related myositis | 1 | (2.44%) |

### Efficacy

Of the 41 patients in this study, 39 patients received at least two tumor assessments, which showed a confirmed ORR of 71.8% among all patients (95% CI: 55.1, 85), including a complete response in 3 patients (7.7%) and partial response in 25 patients (64.1%) as described in Table 8 below; DCR was 92.3%(95% CI: 79.1, 98.4). The cORR for naïve la/mUC patients was at 73.9%. The cORR for HER2 expression (IHC 1+, IHC2+, IHC3+) in la/mUC patients was 77.8%. The ORRs were 100%, 77.8%, 66.7%, and 50% for patients with HER2 (3+), HER2 (2+), HER2 (1+), and HER2 (0), respectively, as shown in Table 9 below. The ORR increased with the high expression of HER2 or PD-L1. The corresponding ORRs were 91.7% in patients with PD-L1 CPS ≥1 and 50% in CPS <1. As well, the mPFS was 9.2 months, and the mOS was not reached.

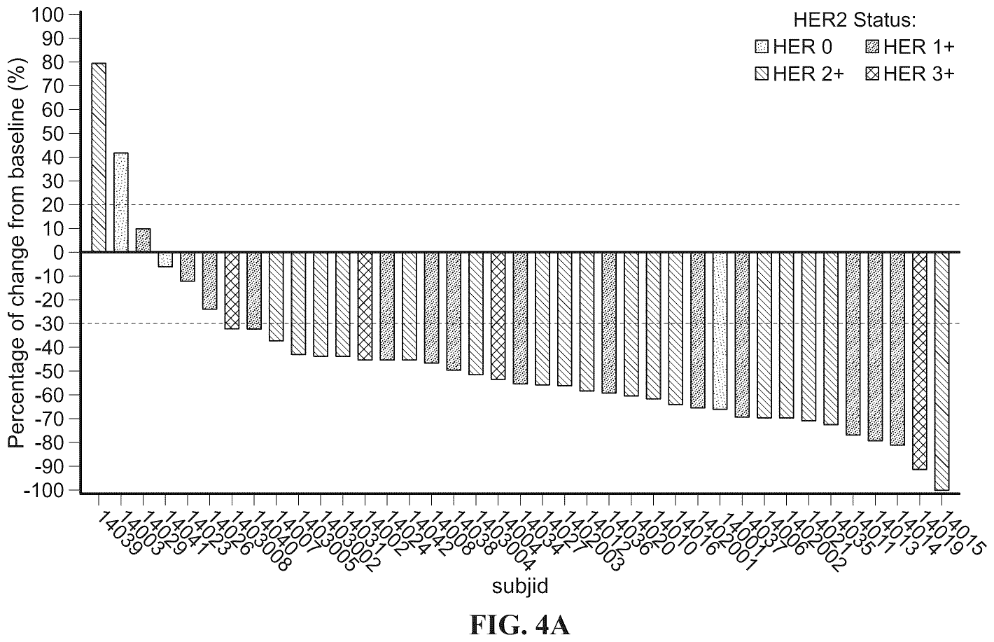

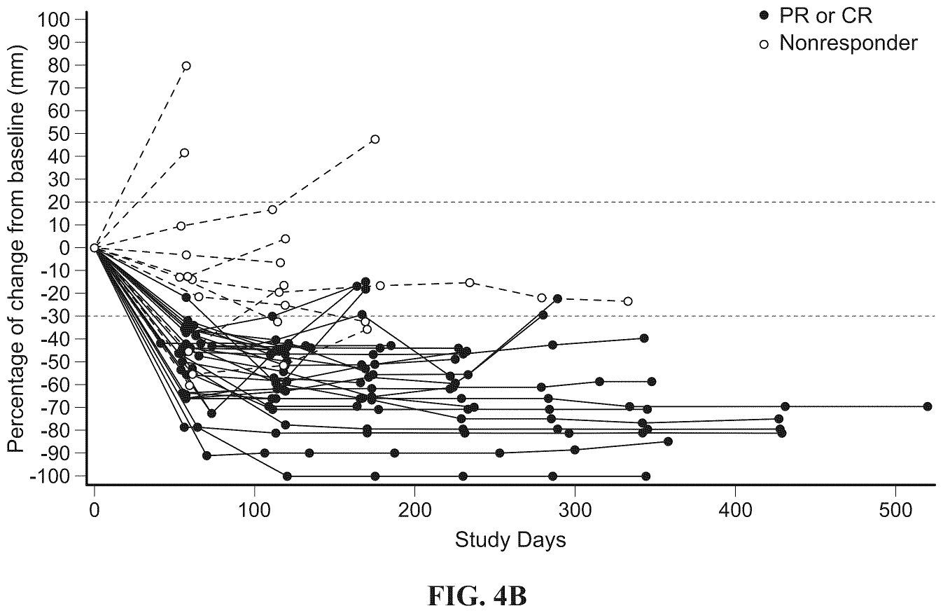

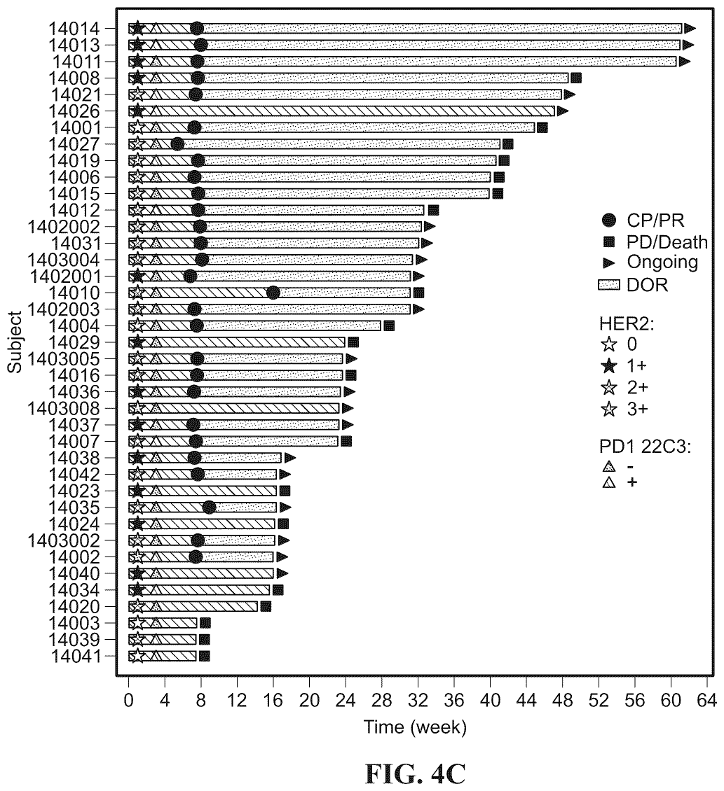

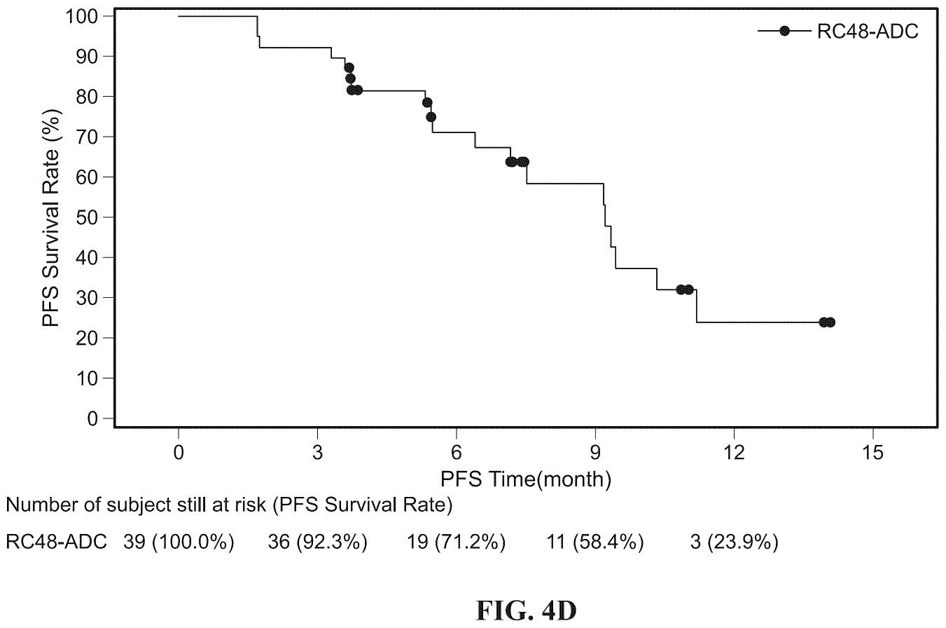

FIGS. 4A-B show percentage of change in target lesions from the measured baseline in patients receiving RC48-ADC and JS001 combination therapy. In FIG. 4A. HER2 status indicates the IHC grade. In FIG. 4B, the sum of diameters of patient target lesions were analyzed for percentage change over time up to 500 days. FIG. 4C shows the efficacy of RC48-ADC and JS001 combination therapy by cORR as time to and duration of response further broken down into response assessment of each subgroup and individual. FIG. 4D shows the percentage of progression free survival over time in patients enrolled in this study. Together, these results indicate that RC48-ADC and JS001 combination therapy yielded an improved patient outcome compared to single treatment alone, in particular with respect to PFS.

TABLE 8
Patient response to RC48-ADC and JS001 combination therapy.

| Overall Population | RC48-ADC (N = 39) | |
|---|---|---|
| Confirmed Objective Response Rate (cORR) | 28 | (71.8%) |
| Complete Remission or Response (CR) | 3 | (7.7%) |
| Partial Remission or Response (PR) | 25 | (64.1%) |
| Stable Disease (SD) | 8 | (20.5%) |
| Progressive Disease (PD) | 3 | (7.7%) |
| Dissease Control Rate (DCR) | 36 | (92.3%) |

| | |
|---|---|
| Duration of Response (DOR) | 8.18 months |

TABLE 9
Patient HER2 and PD-L1 subgroup analysis for cORR.

| Subgroup | cORR (%, 95% CI) | |
|---|---|---|
| Prior Systemic Treatment | | |
| 0 Line (n = 23) | 80 | (44.4, 97.5) |
| 1+ Lines (n = 16) | 75 | (34.9, 96.8) |
| HER2 & PD-L1 Expression | | |
| HER2 IHC (2+/3+), PD-L1(+) (n = 8) | 100 | (29.2, 100) |
| HER2 IHC (2+/3+), PD-L1(−) (n = 16) | 77.8 | (40, 97.2) |
| HER2 IHC (1+), PD-L1(+) (n = 4) | 66.7 | (22.3, 95.7) |
| HER2 IHC (1+), PD-L1(−) (n = 10) | 50 | (1.3, 98.7) |
| HER2 IHC (0), PD-L1(+) (n = 1) | | |
| HER2 IHC (0), PD-L1(−) (n = 2) | 50 | (15.7, 84.3) |

1. (canceled)

2. A method for treating a urothelial cancer patient, comprising administering to the patient an effective amount of an antibody-drug conjugate (ADC) and an immune checkpoint inhibitor,
wherein the antibody-drug conjugate has the structure of the general formula Ab-(L-U)ₙ, wherein Ab represents anti-Her2 (Human epidermal growth factor receptor 2) antibody; L represents a linker, U represents a conjugated cytotoxic molecule; and n is an integer from 1 to 8, and represents the number of cytotoxic molecules bound to each antibody, and wherein:
the antibody comprises a heavy chain variable region and a light chain variable region, wherein the CDR of the heavy chain variable region and/or the CDR of the light chain variable region have the same CDR sequences as Disitamab vedotin;
the linker L comprises Maleimido-Caproyl-Valine-Citrulline-p-Aminobenzyloxy (mc-vc-pAB), wherein the linker is covalently linked to the antibody by means of sulfhydryl conjugation, and the linking site is the interchain disulfide bond site of the antibody;
the cytotoxic molecule U comprises MMAE (monomethyl auristatin E); and
the immune checkpoint inhibitor is a PD-1 antibody or a PD-L1 antibody.

3. The method according to claim 2, wherein the patient is positive for HER2 expression.

4. The method according to claim 2, wherein a sample obtained from urothelial cancer of the patient is HER2 positive.

5. The method according to claim 2, wherein the sample obtained from urothelial cancer of the patient is HER2 positive based on a immunohistochemistry (IHC) assay.

6. The method according toclaim 2, wherein HER2 expression in the sample obtained from urothelial cancer of the patient is IHC 3+ or IHC 2+.

7. The method according to claim 2, wherein the patient is positive for PD-L1 or PD-1 expression.

8. The method according to claim 2, wherein the antibody comprises a heavy chain variable (VH) region and a light chain variable (VL) region;
wherein the VH region comprises an HCDR1 comprising the amino acid sequence of GYTFTDYY (SEQ ID NO:3), an HCDR2 comprising the amino acid sequence of VNPDHGDS (SEQ ID NO:4), and an HCDR3 comprising the amino acid sequence of ARNYLFDH (SEQ ID NO:5); and
wherein the VL region comprises a LCDR1 comprising the amino acid sequence of QDVGTA (SEQ ID NO:6), a LCDR2 comprising the amino acid sequence of WAS (SEQ ID NO:7), and a LCDR3 comprising the amino acid sequence of HQFATYT (SEQ ID NO:8).

9. The method according to claim 2, wherein the antibody comprises a heavy chain variable (VH) region and a light chain variable (VL) region;
wherein the VH region comprises an HCDR1 comprising the amino acid sequence of DYYIH (SEQ ID NO:31), an HCDR2 comprising the amino acid sequence of RVNPDHGDSYYNQKFKD (SEQ ID NO:32), and an HCDR3 comprising the amino acid sequence of ARNYLFDHW (SEQ ID NO:33); and
wherein the VL region comprises a LCDR1 comprising the amino acid sequence of KASQDVGTAVA (SEQ ID NO:34), a LCDR2 comprising the amino acid sequence of WASIRHT (SEQ ID NO:35), and a LCDR3 comprising the amino acid sequence of HQFATYT (SEQ ID NO:8).

10. The method according to claim 2, wherein the antibody is a murine, chimeric, or humanized antibody.

11. The method according to claim 2, wherein the antibody comprises a heavy chain variable (VH) region and a light chain variable (VL) region; wherein the VH region comprises the amino acid sequence of EVQLVQSGAEVKKPGATVKISCKVSGYTFTDYYIHWVQQAPGKGLEWMGRVNPDHGD SYYNQKFKDKATITADKSTDTAYMELSSLRSEDTAVYFCARNYLFDHWGQGTLVTVSS (SEQ ID NO:9); and wherein the VL region comprises the amino acid sequence of DIQMTQSPSSVSASVGDRVTITCKASQDVGTAVAWYQQKPGKAPKLLIYWASIRHTGVP SRFSGSGSGTDFTLTISSLQPEDFATYYCHQFATYTFGGGTKVEIK (SEQ ID NO:10).

12. The method according to claim 2, wherein the antibody is a human IgG antibody.

13. The method according to claim 2, wherein the antibody is a human IgG1, IgG2, and IgG4 antibody.

14. The method according to claim 2, wherein the amino acid sequence of the heavy chain of the antibody is SEQ ID NO:1, and the amino acid sequence of the light chain of the antibody is SEQ ID NO:2.

15. The method according to claim 2, wherein the antibody-drug conjugate is Disitamab vedotin or a biosimilar thereof.

16. The method according to claim 2, wherein the average DAR (i.e., Drug-to-Antibody Ratio) value of the antibody-drug conjugate is any number from 2 to 7.

17. The method according to claim 16, wherein the average DAR value is 4±0.5.

18. The method according to claim 2, wherein the immune checkpoint inhibitor is a PD-1 antibody.

19. The method according to claim 18, wherein the PD-1 antibody is selected from the group consisting of Toripalimab, Dostarlimab, Prolgolimab, Tislelizumab, Camrelizumab, Sintilimab, Cemiplimab, Pembrolizumab, Nivolumab, Penpulimab, Genolimzumab, Zimberelimab, and Balstilimab.

20. The method according to claim 2, wherein the immune checkpoint inhibitor is a PD-L1 antibody.

21. The method according to claim 20, wherein the immune checkpoint inhibitor is a PD-L1 antibody is selected from the group consisting of Durvalumab, Avelumab, Atezolizumab, Envafolimab, and RC98.

22. The method according to claim 2, wherein the patient has previously received one or more prior treatments selected from the group consisting of chemotherapy drugs, targeted therapy, immunotherapy and endocrine therapy.

23. The method according to claim 2, wherein the urothelial cancer patient is selected from the group consisting of a patient with locally advanced urothelial cancer that cannot be surgically resected, a patient with locally advanced or metastatic urothelial cancer, a patient with HER2-positive urothelial cancer, a patient with HER2 positive locally advanced or metastatic urothelial cancer, and a urothelial cancer patient who cannot tolerate platinum-based chemotherapy.

24. The method according to claim 2, wherein the urothelial cancer patient is a patient with unresectable locally advanced or metastatic urothelial carcinoma.

25. The method according to claim 2, wherein the urothelial cancer patient is a patient who is ineligible for or has refused cisplatin based chemotherapy.

26. The method according to claim 2, wherein the urothelial cancer patient is a patient who has progressed after chemotherapy.

27. The method according to claim 2, wherein the urothelial cancer patient is a patient who has experienced disease progression within 12 months of completion of neoadjuvant or adjuvant cisplatin-based chemotherapy.

28. The method according to claim 2, wherein the medicament is administered intranasally, subcutaneously, intradermally, intramuscularly or intravenously.

29. The method according to claim 2, wherein the ADC is administered at a dosage of 1.5 mg/kg or 2.0 mg/kg.

30. The method according to claim 2, wherein the ADC is administered every 2 weeks or 14 days.

31. The method according to claim 2, wherein administration of the antibody-drug conjugate and immune checkpoint inhibitor to the urothelial cancer patient results in progression-free survival (PFS) of greater than 7.5 months.

32.-36. (canceled)


SEQUENCE LISTING (35 sequences)

SEQ ID NO: 1 - protein, 445 aa, synthetic construct
  REGION 1..445: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, synthetic construct
  REGION 1..212: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, synthetic construct
  REGION 1..8: note = Synthetic Construct
GYTFTDYY

SEQ ID NO: 4 - protein, 8 aa, synthetic construct
  REGION 1..8: note = Synthetic Construct
VNPDHGDS

SEQ ID NO: 5 - protein, 8 aa, synthetic construct
  REGION 1..8: note = Synthetic Construct
ARNYLFDH

SEQ ID NO: 6 - protein, 6 aa, synthetic construct
  REGION 1..6: note = Synthetic Construct
QDVGTA

SEQ ID NO: 7 - skipped

SEQ ID NO: 8 - protein, 7 aa, synthetic construct
  REGION 1..7: note = Synthetic Construct
HQFATYT

SEQ ID NO: 9 - protein, 115 aa, synthetic construct
  REGION 1..115: note = Synthetic Construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 10 - protein, 105 aa, synthetic construct
  REGION 1..105: note = Synthetic Construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 11 - protein, 452 aa, synthetic construct
  REGION 1..452: note = Synthetic Construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 219 aa, synthetic construct
  REGION 1..219: note = Synthetic Construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 8 aa, synthetic construct
  REGION 1..8: note = Synthetic Construct
GYTFTDYE

SEQ ID NO: 14 - protein, 8 aa, synthetic construct
  REGION 1..8: note = Synthetic Construct
IESETGGT

SEQ ID NO: 15 - protein, 18 aa, synthetic construct
  REGION 1..18: note = Synthetic Construct
AREGITTVATTYYWYFDV

SEQ ID NO: 16 - protein, 11 aa, synthetic construct
  REGION 1..11: note = Synthetic Construct
QSIVHSNGNTY

SEQ ID NO: 17 - skipped

SEQ ID NO: 18 - protein, 9 aa, synthetic construct
  REGION 1..9: note = Synthetic Construct
FQGSHVPLT

SEQ ID NO: 19 - protein, 125 aa, synthetic construct [sequence omitted]

SEQ ID NO: 20 - protein, 115 aa, synthetic construct
  REGION 1..115: note = Synthetic Construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTV

SEQ ID NO: 21 - protein, 450 aa, synthetic construct
  REGION 1..450: note = Synthetic Construct
QVQLQESGPGLVKPSETLSLTCTVSGFSLSRYSVHWIRQPPGKGLEWLGMIWGVGTTDYNSALKSRLTISKDTSKNQFSLKLSSVTAADTAVYYCARNWGTADYFDYWGQGTTVTVSSASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 220 aa, synthetic construct
  REGION 1..220: note = Synthetic Construct
DIVLTQSPASLAVSPGQRATITCRASKSVHTSGYSYMHWYQQKPGQPPKLLIYLASNLESGVPARFSGSGSGTDFTLTINPVEANDTANYYCQHSGELPYTFGGGTKVEIKRTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 8 aa, synthetic construct
  REGION 1..8: note = Synthetic Construct
GFSLSRYS

SEQ ID NO: 24 - protein, 7 aa, synthetic construct
  REGION 1..7: note = Synthetic Construct
IWGVGTT

SEQ ID NO: 25 - protein, 12 aa, synthetic construct
  REGION 1..12: note = Synthetic Construct
ARNWGTADYFDY

SEQ ID NO: 26 - protein, 10 aa, synthetic construct
  REGION 1..10: note = Synthetic Construct
KSVHTSGYSY

SEQ ID NO: 27 - skipped

SEQ ID NO: 28 - protein, 9 aa, synthetic construct
  REGION 1..9: note = Synthetic Construct
QHSGELPYT

SEQ ID NO: 29 - protein, 120 aa, synthetic construct
  REGION 1..120: note = Synthetic Construct
QVQLQESGPGLVKPSETLSLTCTVSGFSLSRYSVHWIRQPPGKGLEWLGMIWGVGTTDYNSALKSRLTISKDTSKNQFSLKLSSVTAADTAVYYCARNWGTADYFDYWGQGTTVTVSSAS

SEQ ID NO: 30 - protein, 113 aa, synthetic construct
  REGION 1..113: note = Synthetic Construct
DIVLTQSPASLAVSPGQRATITCRASKSVHTSGYSYMHWYQQKPGQPPKLLIYLASNLESGVPARFSGSGSGTDFTLTINPVEANDTANYYCQHSGELPYTFGGGTKVEIKRT

SEQ ID NO: 31 - protein, 5 aa, synthetic construct
  REGION 1..5: note = Synthetic Construct
DYYIH

SEQ ID NO: 32 - protein, 17 aa, synthetic construct
  REGION 1..17: note = Synthetic Construct
RVNPDHGDSYYNQKFKD

SEQ ID NO: 33 - protein, 9 aa, synthetic construct
  REGION 1..9: note = Synthetic Construct
ARNYLFDHW

SEQ ID NO: 34 - protein, 11 aa, synthetic construct
  REGION 1..11: note = Synthetic Construct
KASQDVGTAVA

SEQ ID NO: 35 - protein, 7 aa, synthetic construct
  REGION 1..7: note = Synthetic Construct
WASIRHT